(19) United States
(12) Patent Application Publication
(10) Pub. No.: US 2025/0204827 A1
(43) Pub. Date: Jun. 26, 2025

(54) CONGENIALITY ESTIMATION DEVICE, CONGENIALITY ESTIMATION METHOD, AND NON-TRANSITORY COMPUTER-READABLE MEDIUM

(71) Applicant: Yokogawa Electric Corporation, Tokyo (JP)

(72) Inventors: Kiyomi MURAOKA, Tokyo (JP); Motohiko ISHIDA, Tokyo (JP); Hirotaka YAMASHITA, Tokyo (JP); Takashi TSUBOTA, Tokyo (JP)

(21) Appl. No.: 18/943,625

(22) Filed: Nov. 11, 2024

(30) Foreign Application Priority Data
Dec. 26, 2023 (JP) 2023-219259

(51) Int. Cl. A61B 5/16; A61B 5/372

(52) U.S. Cl. CPC A61B 5/167; A61B 5/372

(57) ABSTRACT

Provided is a congeniality estimation device including: an information acquisition unit that acquires at least one of first electroencephalogram information of a plurality of first target persons with a first attribute or second electroencephalogram information of a plurality of second target persons with a second attribute in the situation where each of the first target persons is interacting with each of the second target persons; and a congeniality estimation unit that estimates congeniality between each of the first target persons and each of the second target persons based on at least one of the first electroencephalogram information or the second electroencephalogram information.

The contents of the following patent application(s) are incorporated herein by reference: NO. 2023-219259 filed in JP on Dec. 26, 2023

### BACKGROUND

### 1. Technical Field

The present invention relates to a congeniality estimation device, a congeniality estimation method, and a non-transitory computer-readable medium.

### 2. Related Art

Patent Document 1 describes “a stress sensing system which can identify the cause of the increased degree of stress” (Abstract). Patent Document 2 describes “generating the profile of the stress level and the stress resistance level of people” (paragraph 0001). Patent Document 3 describes “estimating the emotion of the first baby based on the ratio of frequency component” (claim 1). Patent Document 4 describes “objectively knowing the stress of caregiver related to the work” (Abstract). Patent Document 5 describes “building a team by selecting members from a plurality of candidates” (paragraph 0001). Patent Document 6 describes “predicting the degree of fatigue of users and proposing the timing for a nap” (Abstract).

### PRIOR ART DOCUMENT

### Patent Document

Patent Document 1: Japanese Patent Application Publication No. 2016-209404
Patent Document 2: Japanese Patent No. 6701215
Patent Document 3: Japanese Patent No. 6627059
Patent Document 4: Japanese Patent Application Publication No. 2019-022540
Patent Document 5: Japanese patent No. 6781012
Patent Document 6: Japanese Patent Application Publication No. 2021-043480

### BRIEF DESCRIPTION OF THE DRAWINGS

FIG. 1 is a diagram showing one example of a situation in which a first target person 110 with a first attribute is interacting with a second target person 120 with a second attribute.

FIG. 2 is a diagram showing another example of a situation in which the first target person 110 is interacting with the second target person 120.

FIG. 3 is a diagram showing another example of a situation in which the first target person 110 is interacting with the second target person 120.

FIG. 4 is a diagram showing another example of a situation in which the first target person 110 is interacting with the second target person 120.

FIG. 5 is a diagram showing another example of a situation in which the first target person 110 is interacting with the second target person 120.

FIG. 6 is a diagram showing one example of a combination of a plurality of first target persons 110 and a plurality of second target persons 120.

FIG. 7 is a block diagram showing one example of a congeniality estimation device 100 according to one embodiment of the present invention.

FIG. 8 is a diagram showing one example of an information acquisition unit 10.

FIG. 9 is a diagram showing one example of a state S1 of the first target person 110.

FIG. 10 is a diagram showing one example of a state S2 of the second target person 120.

FIG. 11 is a diagram showing one example of a congeniality Co.

FIG. 12 is a diagram showing one example of a combination of the plurality of groups including two or more first target persons 110 and the plurality of second target persons 120.

FIG. 13 is a flowchart showing one example of a congeniality estimation method according to one embodiment of the present invention.

FIG. 14 is a diagram showing one example of a computer 2200 in which the congeniality estimation device 100 according to one embodiment of the present invention may be wholly or partially embodied.

### DESCRIPTION OF EXEMPLARY EMBODIMENTS

Hereinafter, the present invention will be described through embodiments of the invention, but the following embodiments do not limit the invention according to the claims. In addition, not all of the combinations of features described in the embodiments are essential to the solution of the invention.

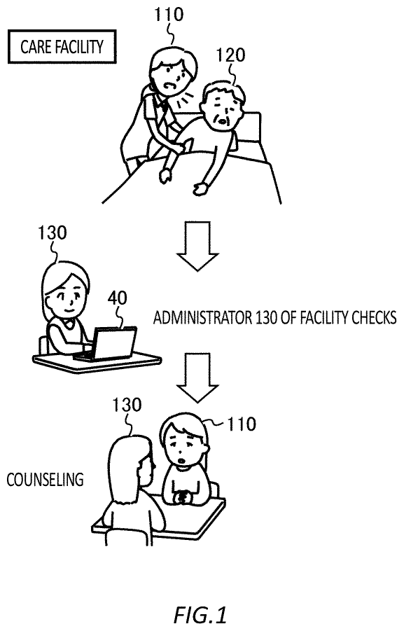

FIG. 1 is a diagram showing one example of a situation in which a first target person 110 with a first attribute is interacting with a second target person 120 with a second attribute. In the present specification, the first target person 110 interacting with the second target person 120 refers to the first target person 110 mentally interacting with the second target person 120 through communication. The first attribute refers to the nature or characteristics of the first target person 110 that has a high probability of affecting the state S2 of the second target person 120 (described below). The second attribute refers to the nature or characteristics of the second target person 120 that has a high probability of affecting the state S2 (described below) by interacting with the first target person 110.

The second target person 120 is a person paired with the first target person 110. In the example of FIG. 1, the first target person 110 is a caregiver and the second target person 120 is a care recipient. For example, if the first target person 110 is a superior, the second target person 120 is a subordinate. For example, if the first target person 110 is a physician, the second target person 120 is a patient. For example, if the first target person 110 is a manager, the second target person 120 is a player.

The presentation unit 40 presents a congeniality Co (described below) between the first target person 110 and the second target person 120. As described below, the congeniality Co is estimated based on at least one of a first electroencephalogram information Ib1 (described below) or a second electroencephalogram information Ib2 (described below). The presentation unit 40 is, for example, a tablet, a display, a monitor, or the like. The administrator 130 may be a user of the congeniality estimation device 100 (described below). In the example of FIG. 1, the administrator 130 is an administrator of a care facility. In the example of FIG. 1, the administrator 130 confirms the congeniality Co (described below) between the first target person 110 and the second target person 120 based on the congeniality Co (described below) presented by the presentation unit 40.

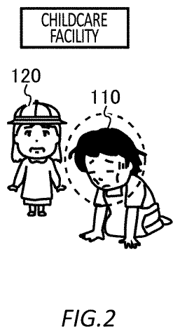

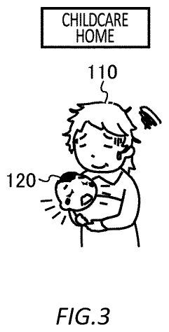

FIG. 2 and FIG. 3 are diagrams showing another example of a situation in which the first target person 110 is interacting with the second target person 120. In the example of FIG. 2, the first target person 110 is a childcare provider and the second target person 120 is a childcare recipient. In the example of FIG. 3, the first target person 110 is a mother and the second target person 120 is a child of the mother.

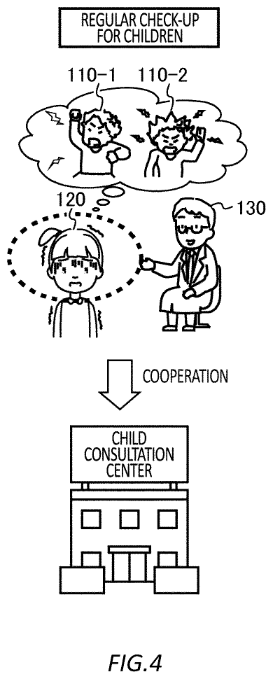

FIG. 4 is a diagram showing another example of a situation in which the first target person 110 is interacting with the second target person 120. In the example of FIG. 4, the first target person 110-1 is a father, the first target person 110-2 is a mother, and the second target person 120 is a child of the father and the mother. In the example of FIG. 4, the second target person 120 is recalling the situation in which it is interacting with the first target person 110. In the example of FIG. 4, the administrator 130 is a physician.

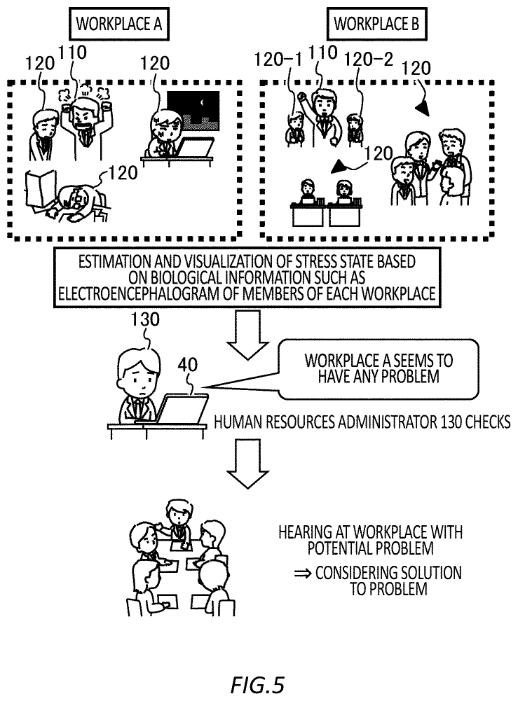

FIG. 5 is a diagram showing another example of a situation in which the first target person 110 is interacting with the second target person 120. In the example of FIG. 5, the first target person 110 is a superior and the second target person 120 is a subordinate. A work place A in FIG. 5 is one example of a situation in which the first target person 110 is reprimanding the second target person 120. A work place B in FIG. 5 is one example of a situation in which the first target person 110 is encouraging the second target person 120. In the example of FIG. 5, the administrator 130 is a human resources administrator of the company having the work place A and the work place B.

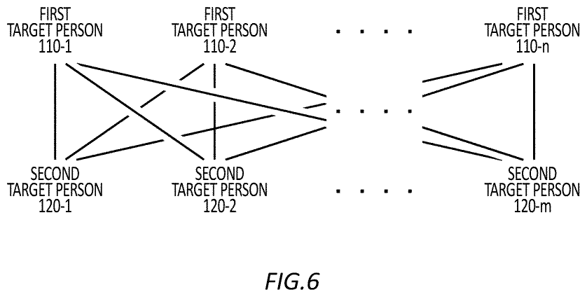

FIG. 6 is a diagram showing one example of a combination of the plurality of first target persons 110 and the plurality of second target persons 120. In the present example, there are n individuals of the first target person 110 (the first target person 110-1 to the first target person 110-n) and there are m individuals of the second target person 120 (the second target person 120-1 to the second target person 120-m). Each of the first target person 110-1 to the first target person 110-n interacts with each of the second target person 120-1 to the second target person 120-m, respectively. FIG. 6 shows the combination of each of the plurality of first target persons 110 and each of the plurality of second target persons 120 using solid lines.

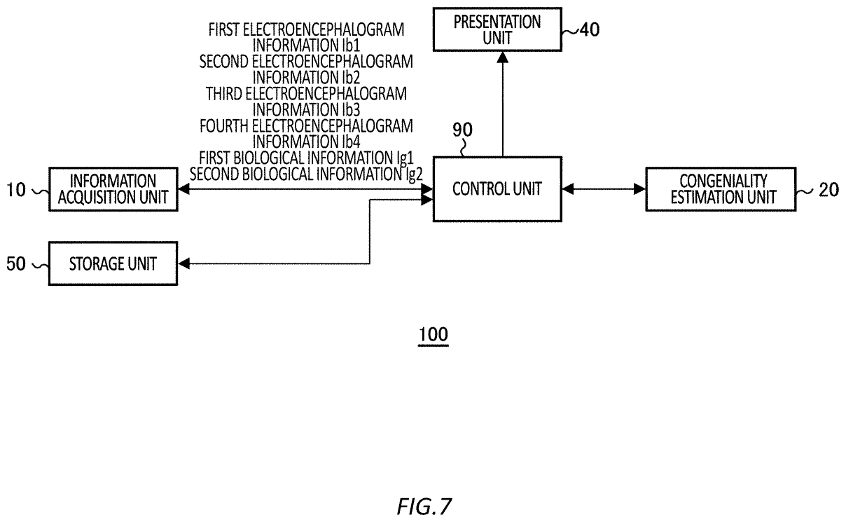

FIG. 7 is a block diagram showing one example of a congeniality estimation device 100 according to one embodiment of the present invention. The congeniality estimation device 100 includes an information acquisition unit 10 and a congeniality estimation unit 20. The congeniality estimation device 100 may include a presentation unit 40, a storage unit 50, and a control unit 90.

The congeniality estimation device 100 may be partially or wholly implemented using a computer. The control unit 90 may be a Central Processing Unit (CPU) of the computer. If the congeniality estimation device 100 is implemented using a computer, the computer may have an evaluation assisting program installed to enable the computer to function as the congeniality estimation device 100 or may have an Information processing program installed to perform the Information processing method described below.

The information acquisition unit 10 acquires the electroencephalogram information of at least one of the first target person 110 or the second target person 120 in a situation in which each of the plurality of first target persons 110 with the first attribute are interacting with each of the plurality of second target persons 120 with the second attribute. The electroencephalogram information of the first target person 110 and the electroencephalogram information of the second target person 120, in a situation in which the first target person 110 is interacting with the second target person 120, are referred to as first electroencephalogram information Ib1 and second electroencephalogram information Ib2, respectively. The information acquisition unit 10 may acquire at least one of the first electroencephalogram information Ib1 or the second electroencephalogram information Ib2 in a situation in which each of the first target person 110-1 to the first target person 110-n is interacting with each of the second target person 120-1 to the second target person 120-m.

The information acquisition unit 10 may acquire the electroencephalogram information of at least one of the first target person 110 or the second target person 120 before each of the plurality of first target persons 110 interacts with each of the plurality of second target persons 120. The electroencephalogram information of the first target person 110 and the electroencephalogram information of the second target person 120, before the first target person 110 interacts with the second target person 120, are referred to as the third electroencephalogram information Ib3 and the fourth electroencephalogram information Ib4, respectively.

The first electroencephalogram information Ib1 and the third electroencephalogram information Ib3 may be the information for reproducing, at least partially, the temporal waveform of the electroencephalogram of the first target person 110. The same may apply to the second electroencephalogram information Ib2 and the fourth electroencephalogram information Ib4. The first electroencephalogram information Ib1 and the third electroencephalogram information Ib3 may include the data in which the temporal waveform of the electroencephalogram is sampled, may include the data indicating the magnitude of the frequency component of the electroencephalogram in one or more frequencies, or may include other data. The same may apply to the second electroencephalogram information Ib2 and the fourth electroencephalogram information Ib4. For example, the first electroencephalogram information Ib1 to the fourth electroencephalogram information Ib4 include the data indicating the magnitude of at least one of the following components: a delta wave (less than 4 Hz), a theta wave (greater than or equal to 4 Hz and less than 8 Hz), an alpha wave (greater than or equal to 8 Hz and less than 14 Hz), a beta wave (greater than or equal to 14 Hz and less than 26 Hz) or a gamma wave (greater than or equal to 26 Hz and less than 40 Hz).

The alpha wave is sometimes further classified into low alpha wave (greater than or equal to 8 Hz and less than 10 Hz), medium alpha wave (greater than or equal to 10 Hz and less than 12 Hz), and high alpha wave (greater than or equal to 12 Hz and less than 14 Hz), according to the frequency band. The electroencephalogram information Ib may include the data indicating the magnitude of at least one of the low alpha wave, the medium alpha wave, or the high alpha wave.

The beta wave is sometimes further classified into low beta wave (greater than or equal to 14 Hz and less than 18 Hz) and high beta wave (greater than or equal to 18 Hz and less than 26 Hz) according to the frequency band. The electroencephalogram information Ib may include the data indicating the magnitude of at least one of the low beta wave or the high beta wave.

The first electroencephalogram information Ib1 and the third electroencephalogram information Ib3 may include the information of the temporal waveform of one or more electroencephalograms, which are measured at one or more positions in the head region including the head and the face of the first target person 110. For example, the first electroencephalogram information Ib1 and the third electroencephalogram information Ib3 may be acquired by measuring the temporal waveform of the electric potential of the electrode arranged at regular intervals near the scalp of the first target person 110 as in the international 10-20 system or may be acquired by another method. The same may apply to the second electroencephalogram information Ib2 and the fourth electroencephalogram information Ib4. A plurality of electrodes may not be arranged on the scalp at regular intervals. The electrode may be provided on the wearable appliance worn on the head region of the first target person 110, such as a headgear, a headphone, an earphone, glasses, or the like. The first electroencephalogram information Ib1 and the third electroencephalogram information Ib3 may be the information that is of the electric signal at the electrode embedded in the body of the first target person 110 and that is acquired via wireless communication. The same may apply to the second electroencephalogram information Ib2 and the fourth electroencephalogram information Ib4.

A total amplitude As is defined as the sum of the amplitudes of the alpha wave, the beta wave, the theta wave, the gamma wave, and the delta wave at a certain timing. As one example, it may be inferred that the first target person 110 is in the sleeping state if the proportion of the amplitude of the delta wave of the first target person 110 to the total amplitude As is greater than any of the proportion of the amplitude of the alpha wave to the total amplitude As, the proportion of the amplitude of the beta wave to the total amplitude As, the proportion of the amplitude of the theta wave to the total amplitude As, and the proportion of the amplitude of the gamma wave to the total amplitude As. The same applies to the second target person 120.

The congeniality estimation unit 20 estimates the congeniality between each of the first target persons 110 and each of the second target persons 120 based on at least one of the first electroencephalogram information Ib1 or the second electroencephalogram information Ib2. The congeniality is referred to as the congeniality Co. The presentation unit 40 may present the congeniality Co to the administrator 130. As a result, the administrator 130 can learn the congeniality Co between the first target person 110 and the second target person 120. By learning the congeniality Co, the administrator 130 easily performs treatments such as counseling for the first target person 110 or the second target person 120.

The congeniality Co may have a plurality of grades. One grade of the congeniality Co is referred to as a grade 1 and another grade is referred to as a grade 2. It is assumed that the congeniality Co of the grade 1 is better than the congeniality Co of the grade 2.

As one example, if the proportion of the amplitude of the theta wave to the total amplitude As in the second electroencephalogram information Ib2 is greater than the proportion of the amplitude of the theta wave to the total amplitude As in the fourth electroencephalogram information Ib4, the congeniality estimation unit 20 may estimate that the congeniality Co between the first target person 110 and the second target person 120 is in the grade 2 (in the present example, the congeniality Co is poor). If the proportion of the amplitude of the theta wave to the total amplitude As in the second electroencephalogram information Ib2 is greater than the proportion of the amplitude of the theta wave to the total amplitude As in the fourth electroencephalogram information Ib4, the second target person 120 may be in the state with increased fatigue or drowsiness. Therefore, the congeniality estimation unit 20 may estimate that the congeniality Co is in the grade 2 (in the present example, the congeniality Co is poor).

Similarly, if the proportion of the amplitude of the theta wave to the total amplitude As in the first electroencephalogram information Ib1 is greater than the proportion of the amplitude of the theta wave to the total amplitude As in the third electroencephalogram information Ib3, the congeniality estimation unit 20 may estimate that the congeniality Co between the first target person 110 and the second target person 120 is in the grade 2 (in the present example, the congeniality Co is poor).

As one example, if the proportion of the sum of the amplitude of the low alpha wave and the amplitude of the medium alpha wave to the total amplitude As in the second electroencephalogram information Ib2 is greater than the proportion of the sum of the amplitude of the low alpha wave and the amplitude of the medium alpha wave to the total amplitude As in the fourth electroencephalogram information Ib4, the congeniality estimation unit 20 may estimate that the congeniality Co between the first target person 110 and the second target person 120 is in the grade 1 (in the present example, the congeniality Co is good). If the proportion of the sum of the amplitude of the low alpha wave and the amplitude of the medium alpha wave to the total amplitude As in the second electroencephalogram information Ib2 is greater than the proportion of the sum of the amplitude of the low alpha wave and the amplitude of the medium alpha wave to the total amplitude As in the fourth electroencephalogram information Ib4, the second target person 120 may be in the state with the increased degree of relaxation. Therefore, the congeniality estimation unit 20 may estimate that the congeniality Co is in the grade 1 (in the present example, the congeniality Co is good).

Similarly, if the proportion of the sum of the amplitude of the low alpha wave and the amplitude of the medium alpha wave to the total amplitude As in the first electroencephalogram information Ib1 is greater than the proportion of the sum of the amplitude of the low alpha wave and the amplitude of the medium alpha wave to the total amplitude As in the third electroencephalogram information Ib3, the congeniality estimation unit 20 may estimate that the congeniality Co between the first target person 110 and the second target person 120 is in the grade 1 (in the present example, the congeniality Co is good).

As one example, if the proportion of the sum of the amplitude of the high alpha wave and the amplitude of the low beta wave to the total amplitude As in the second electroencephalogram information Ib2 is greater than the proportion of the sum of the amplitude of the high alpha wave and the amplitude of the low beta wave to the total amplitude As in the fourth electroencephalogram information Ib4, the congeniality estimation unit 20 may estimate that the congeniality Co between the first target person 110 and the second target person 120 is in the grade 1 (in the present example, the congeniality Co is good). If the proportion of the sum of the amplitude of the high alpha wave and the amplitude of the low beta wave to the total amplitude As in the second electroencephalogram information Ib2 is greater than the proportion of the sum of the amplitude of the high alpha wave and the amplitude of the low beta wave to the total amplitude As in the fourth electroencephalogram information Ib4, the second target person 120 may be in the state with a good balance between relaxation and concentration. The state with a good balance between the relaxation and the concentration is a so-called a state of immersion. Therefore, the congeniality estimation unit 20 may estimate that the congeniality Co is in the grade 1 (in the present example, the congeniality Co is good).

Similarly, if the proportion of the sum of the amplitude of the high alpha wave and the amplitude of the low beta wave to the total amplitude As in the first electroencephalogram information Ib1 is greater than the proportion of the sum of the amplitude of the high alpha wave and the amplitude of the low beta wave to the total amplitude As in the third electroencephalogram information Ib3, the congeniality estimation unit 20 may estimate that the congeniality Co between the first target person 110 and the second target person 120 is in the grade 1 (in the present example, the congeniality Co is good).

The first electroencephalogram information Ib1 and the second electroencephalogram information Ib2 may reflect the congeniality Co between the first target person 110 and the second target person 120. The congeniality estimation device 100 estimates the congeniality Co between the first target person 110 and the second target person 120 based on at least one of the first electroencephalogram information Ib1 or the second electroencephalogram information Ib2. Therefore, the user of the congeniality estimation device 100 (for example, the administrator 130) can learn the congeniality Co between the first target person 110 and the second target person 120.

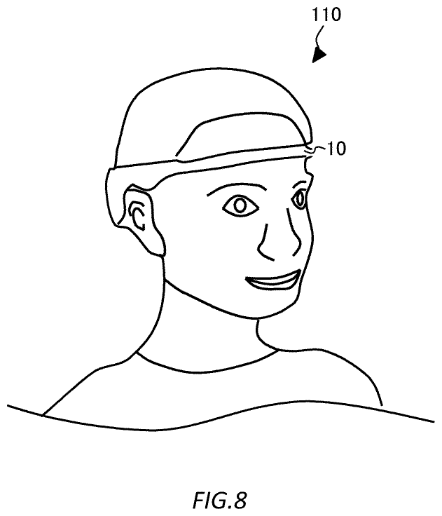

FIG. 8 is a diagram showing one example of an information acquisition unit 10. The information acquisition unit 10 may have an electroencephalograph that can measure the first electroencephalogram information Ib1 and the third electroencephalogram information Ib3 or may have a transmitter that acquires the first electroencephalogram information Ib1 and the third electroencephalogram information Ib3 measured by an external electroencephalograph. The information acquisition unit 10 of the present example is an electroencephalograph of a headgear type. The information acquisition unit 10 may be an electroencephalograph of an earphone type. The first target person 110 may interact with the second target person 120 while wearing the electroencephalograph of a headgear type or an earphone type. Thus, the information acquisition unit 10 acquires the first electroencephalogram information Ib1.

Similarly, the information acquisition unit 10 may have an electroencephalograph that can measure the second electroencephalogram information Ib2 and the fourth electroencephalogram information Ib4 or may have a transmitter that acquires the second electroencephalogram information Ib2 and the fourth electroencephalogram information Ib4 measured by an external electroencephalograph. The second target person 120 may interact with the first target person 110 while wearing the electroencephalograph of a headgear type or an earphone type. Thus, the information acquisition unit 10 acquires the second electroencephalogram information Ib2.

If the information acquisition unit 10 is the electroencephalograph of the headgear type, the control unit 90 may not be contained in the housing of the headgear. The first electroencephalogram information Ib1 to the fourth electroencephalogram information Ib4 acquired by the information acquisition unit 10 may be transmitted to the control unit 90 in a wireless manner.

The information acquisition unit 10 may further acquire the biological information of at least one of the first target person 110 or the second target person 120 in the situation in which each of the first target persons 110 is interacting with each of the second target persons 120. The biological information of the first target person 110 is referred to as first biological information Ig1. The biological information of the second target person 120 is referred to as second biological information Ig2.

The first biological information Ig1 and the second biological information Ig2 may include at least one of the heartbeat information, the perspiration amount information, or the body temperature information of the first target person 110 and the second target person 120, respectively. The first biological information Ig1 and the second biological information Ig2 may be acquired via the sensors provided on the wearable appliances worn on the first target person 110 and the second target person 120, respectively.

The congeniality estimation unit 20 may estimate the congeniality Co based on at least one of the first electroencephalogram information Ib1 or the second electroencephalogram information Ib2 and at least one of the first biological information Ig1 or the second biological information Ig2. The congeniality estimation unit 20 may estimate the congeniality Co based on the change from the third electroencephalogram information Ib3 to the first electroencephalogram information Ib1 and the first biological information Ig1. The congeniality estimation unit 20 may estimate the congeniality Co based on the change from the fourth electroencephalogram information Ib4 to the second electroencephalogram information Ib2 and the second biological information Ig2.

The magnitude of the first power spectrum and the magnitude of the second power spectrum in the heartbeat of the first target person 110 are referred to as LF1 and HF1, respectively. The magnitude of the first power spectrum and the magnitude of the second power spectrum in the heartbeat of the second target person 120 are referred to as LF2 and HF2, respectively. A frequency band of the second power spectrum is a higher frequency band than a frequency band of the first power spectrum. The frequency band of the first power spectrum and the frequency band of the second power spectrum may not overlap each other. The frequency band of the first power spectrum is, for example, 0.04 Hz to 0.15 Hz. The frequency band of the second power spectrum is, for example, 0.15 Hz to 0.4 Hz.

The change from the proportion of the amplitude of the electroencephalogram in a predetermined frequency band to the total amplitude As in the third electroencephalogram information Ib3 to the proportion of the amplitude of the electroencephalogram in the predetermined frequency band to the total amplitude As in the first electroencephalogram information Ib1 is referred to as a change C1. The congeniality estimation unit 20 may estimate the congeniality Co based on the change C1 and the ratio of LF1 to HF1 (LF1/HF1). The change from the proportion of the amplitude of the electroencephalogram in a predetermined frequency band to the total amplitude As in the fourth electroencephalogram information Ib4 to the proportion of the amplitude of the electroencephalogram in the predetermined frequency band to the total amplitude As in the second electroencephalogram information Ib2 is referred to as a change C2. The congeniality estimation unit 20 may estimate the congeniality Co based on the change C2 and the ratio of LF2 to HF2 (LF2/HF2).

The ratio of LF1 to HF1 (LF1/HF1) in a situation in which the first target person 110 is interacting with the second target person 120 is referred to as a ratio Rag1. The predetermined threshold of the ratio Rag1 is referred to as a threshold Pth1. The ratio of LF2 to HF2 (LF2/HF2) in the situation in which the first target person 110 is interacting with the second target person 120 is referred to as a ratio Rag2. The predetermined threshold of the ratio Rag2 is referred to as a threshold Pth2.

As one example, if the proportion of the sum of the amplitude of the high beta wave and the amplitude of the gamma wave to the total amplitude As in the first electroencephalogram information Ib1 is greater than the proportion of the sum of the amplitude of the high beta wave and the amplitude of the gamma wave to the total amplitude As in the third electroencephalogram information Ib3 and the ratio of LF1 to HF1 (LF1/HF1) is greater than or equal to the threshold Pth1, it may be inferred that the irritated state, nervous state, or stress state of the first target person 110 is increased. If the ratio of LF1 to HF1 (LF1/HF1) is greater than or equal to the threshold Pth1, it may be determined that the first target person 110 is in a state where the sympathetic nerve is dominant over the parasympathetic nerve. If the ratio of LF1 to HF1 (LF1/HF1) is less than the threshold Pth1, it may be determined that the first target person 110 is in a state where the parasympathetic nerve is dominant over the sympathetic nerve. The threshold Pth1 may be 2, may be 3, may be 4, or may be 5.

Similarly, if the proportion of the sum of the amplitude of the high beta wave and the amplitude of the gamma wave to the total amplitude As in the second electroencephalogram information Ib2 is greater than the proportion of the sum of the amplitude of the high beta wave and the amplitude of the gamma wave to the total amplitude As in the fourth electroencephalogram information Ib4 and the ratio of LF2 to HF2 (LF2/HF2) is greater than or equal to the threshold Pth2, it may be inferred that the irritated state, nervous state, or stress state of the second target person 120 is increased.

As one example, if the proportion of the sum of the amplitude of the high beta wave and the amplitude of the gamma wave to the total amplitude As in the first electroencephalogram information Ib1 is greater than the proportion of the sum of the amplitude of the high beta wave and the amplitude of the gamma wave to the total amplitude As in the third electroencephalogram information Ib3 and the ratio of LF1 to HF1 (LF1/HF1) is less than the threshold Pth1, it may be inferred that the excited state of the first target person 110 is increased. Similarly, if the proportion of the sum of the amplitude of the high beta wave and the amplitude of the gamma wave to the total amplitude As in the second electroencephalogram information Ib2 is greater than the proportion of the sum of the amplitude of the high beta wave and the amplitude of the gamma wave to the total amplitude As in the fourth electroencephalogram information Ib4 and the ratio of LF2 to HF2 (LF2/HF2) is less than the threshold Pth2, it may be inferred that the excited state of the second target person 120 is increased.

The congeniality estimation unit 20 may estimate the congeniality Co based on the magnitude relationship between the ratio Rag1 and the threshold Pth1, and the change C1. If the congeniality Co has a plurality of grades, the congeniality estimation unit 20 may estimate, based on the magnitude relationship between the ratio Rag1 and the threshold Pth1 and the change C1, that the congeniality Co is in one grade among the plurality of grades. As described above, one grade of the congeniality Co is referred to as a grade 1 and another grade is referred to as a grade 2. It is assumed that the congeniality Co of the grade 1 is better than the congeniality Co of the grade 2.

The proportion of the sum of the amplitude of the high beta wave and the amplitude of the gamma wave to the total amplitude As in the third electroencephalogram information Ib3 is referred to as a proportion Ra3. The proportion of the sum of the amplitude of the high beta wave and the amplitude of the gamma wave to the total amplitude As in the first electroencephalogram information Ib1 is referred to as a proportion Ra1.

If the proportion Ra1 is greater than the proportion Ra3 and the ratio Rag1 is greater than or equal to the threshold Pth1, the congeniality estimation unit 20 may estimate that the congeniality Co between the first target person 110 and the second target person 120 is in the grade 2. If the proportion Ra1 is greater than the proportion Ra3 and the ratio Rag1 is greater than or equal to the threshold Pth1, the first target person 110 may be in the state where the alertness toward the second target person 120 is increased. Thus, the congeniality estimation unit 20 may estimate that the congeniality Co is in the grade 2 (in the present example, the congeniality Co is poor).

If the proportion Ra1 is greater than the proportion Ra3 and the ratio Rag1 is less than the threshold Pth1, the congeniality estimation unit 20 may estimate that the congeniality Co between the first target person 110 and the second target person 120 is in the grade 1. If the proportion Ra1 is greater than the proportion Ra3 and the ratio Rag1 is less than the threshold Pth1, the first target person 110 may be in the state where the degree of excitation toward the second target person 120 is increased. Therefore, the congeniality estimation unit 20 may estimate that the congeniality Co is in the grade 1 (in the present example, the congeniality Co is good).

The congeniality estimation unit 20 may estimate the congeniality Co based on the magnitude relationship between the ratio Rag 2 and the threshold Pth2, and the change C2. The congeniality estimation unit 20 may estimate that the congeniality Co is in one grade among the plurality of grades based on the magnitude relationship of the ratio Rag 2 and the threshold Pth2, and the change C2.

The proportion of the sum of the amplitude of the high beta wave and the amplitude of the gamma wave to the total amplitude As in the fourth electroencephalogram information Ib4 is referred to as a proportion Ra 4. The proportion of the sum of the amplitude of the high beta wave and the amplitude of the gamma wave to the total amplitude As in the second electroencephalogram information Ib2 is referred to as a proportion Ra 2.

If the proportion Ra 2 is greater than the proportion Ra 4 and the ratio Rag 2 is greater than or equal to the threshold Pth2, the congeniality estimation unit 20 may estimate that the congeniality Co between the first target person 110 and the second target person 120 is in the grade 2. If the proportion Ra 2 is greater than the proportion Ra 4 and the ratio Rag 2 is greater than or equal to the threshold Pth2, the second target person 120 may be in the state where the alertness toward the first target person 110 is increased. Thus, the congeniality estimation unit 20 may estimate that the congeniality Co is in the grade 2 (in the present example, the congeniality Co is poor).

If the proportion Ra 2 is greater than the proportion Ra 4 and the ratio Rag 2 is less than the threshold Pth2, the congeniality estimation unit 20 may estimate that the congeniality Co between the first target person 110 and the second target person 120 is in the grade 1. If the proportion Ra 2 is greater than the proportion Ra 4 and the ratio Rag 2 is less than the threshold Pth2, the second target person 120 may be in the state where the degree of excitation toward the first target person 110 is increased. Therefore, the congeniality estimation unit 20 may estimate that the congeniality Co is in the grade 1 (in the present example, the congeniality Co is good).

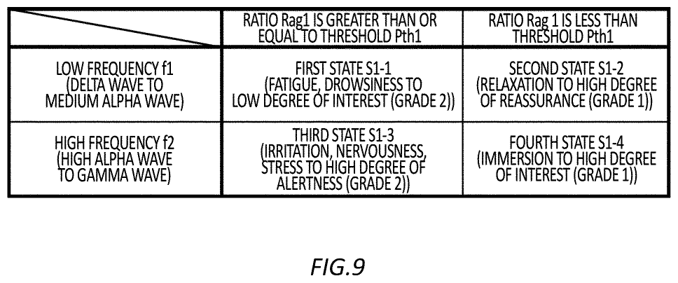

FIG. 9 is a diagram showing one example of the state S1 of the first target person 110. The congeniality estimation unit 20 may estimate the state S1 of the first target person 110 based on the first electroencephalogram information Ib1 and the first biological information Ig1. The state S1 may include a plurality of states of the first target person 110 (the first state S1-1 to the nth state S1-n). In the present example, the state S1 includes four states of the first target person 110 (the first state S1-1 to the fourth state S1-4). The congeniality estimation unit 20 may estimate one of the plurality of states S1 of the first target person 110 based on the first electroencephalogram information Ib1 and the first biological information Ig1.

In FIG. 9, the electroencephalogram of the low frequency f1 refers to at least one of the delta wave, the theta wave, the low alpha wave, or the medium alpha wave and the electroencephalogram of the high frequency f2 refers to at least one of the high alpha wave, the low beta wave, the high beta wave, or the gamma wave.

The amplitude of the electroencephalogram that is in the first electroencephalogram information Ib1 and that is in a predetermined frequency band is referred to as an amplitude Af2. The amplitude of the electroencephalogram that is in the third electroencephalogram information Ib3 and that is in a predetermined frequency band is referred to as an amplitude Af1. The electroencephalogram in the predetermined frequency band may be at least one of the low alpha wave, the medium alpha wave, the high alpha wave, the low beta wave, the high beta wave, the gamma wave, or the theta wave.

In the present example, the first state S1-1 is the state S1 of the first target person 110 in the case where the proportion of the amplitude Af2 to the total amplitude As is greater than the proportion of the amplitude Af1 to the total amplitude As in the electroencephalogram of the low frequency f1 and the ratio of LF1 to HF1 (LF1/HF1) (the ratio Rag1 described above) is greater than or equal to the threshold Pth1. If the first target person 110 is in the first state S1-1, it may be inferred that the first target person 110 is in the state where fatigue or drowsiness is increased. Therefore, it may be inferred that the degree of interest of the first target person 110 toward the second target person 120 is decreased. Therefore, the congeniality estimation unit 20 may estimate that the congeniality Co between the first target person 110 and the second target person 120 is in the grade 2 (in the present example, the congeniality Co is poor).

In the present example, the second state S1-2 is the state S1 of the first target person 110 in the case where the proportion of the amplitude Af2 to the total amplitude As is greater than the proportion of the amplitude Af1 to the total amplitude As in the electroencephalogram of the low frequency f1 and the ratio Rag1 is less than the threshold Pth1. If the first target person 110 is in the second state S1-2, it may be inferred that the first target person 110 is in the state where the degree of relaxation is increased. Therefore, it may be inferred that the degree of reassurance of the first target person 110 toward the second target person 120 is increased. Thus, the congeniality estimation unit 20 may estimate that the congeniality Co between the first target person 110 and the second target person 120 is in the grade 1 (in the present example, the congeniality Co is good).

In the present example, the third state S1-3 is the state S1 of the first target person 110 in the case where the proportion of the amplitude Af2 to the total amplitude As is greater than the proportion of the amplitude Af1 to the total amplitude As in the electroencephalogram of the high frequency f2 and the ratio Rag1 is greater than or equal to the threshold Pth1. If the first target person 110 is in the third state S1-3, it may be inferred that the first target person 110 is in the state where the irritation, nervousness, or stress is increased. Therefore, it may be inferred that the degree of alertness of the first target person 110 toward the second target person 120 is increased. Therefore, the congeniality estimation unit 20 may estimate that the congeniality Co between the first target person 110 and the second target person 120 is in the grade 2 (in the present example, the congeniality Co is poor).

In the present example, the fourth state S1-4 is the state S1 of the first target person 110 in the case where the proportion of the amplitude Af2 to the total amplitude As is greater than the proportion of the amplitude Af1 to the total amplitude As in the electroencephalogram of the high frequency f2 and the ratio Rag1 is less than the threshold Pth1. If the first target person 110 is in the fourth state S1-4, it may be inferred that the first target person 110 is in the state where the degree of preoccupation is increased. Therefore, it may be inferred that the degree of interest of the first target person 110 toward the second target person 120 is increased. Thus, the congeniality estimation unit 20 may estimate that the congeniality Co between the first target person 110 and the second target person 120 is in the grade 2 (in the present example, the congeniality Co is good).

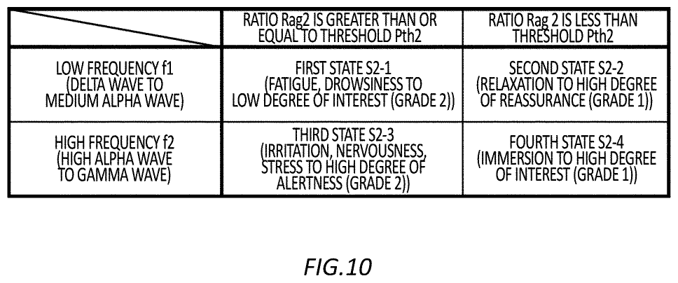

FIG. 10 is a diagram showing one example of a state S2 of the second target person 120. The congeniality estimation unit 20 may estimate the state S2 of the second target person 120 based on the second electroencephalogram information Ib2 and the second biological information Ig2. The state S2 may include a plurality of states (the first state S2-1 to the nth state S2-n) of the second target person 120. In the present example, the state S2 includes the four states (the first state S2-1 to the fourth state S2-4) of the second target person 120. The congeniality estimation unit 20 may estimate one of the plurality of states S2 of the second target person 120 based on the second electroencephalogram information Ib2 and the second biological information Ig2.

As in the case of FIG. 9, if the second target person 120 is in the first state S2-1, it may be inferred that the second target person 120 is in the state where the fatigue or drowsiness is increased. Therefore, the congeniality estimation unit 20 may estimate that the congeniality Co between the first target person 110 and the second target person 120 is in the grade 2 (in the present example, the congeniality Co is poor). If the second target person 120 is in the second state S2-2, It may be inferred that the second target person 120 is in the state where the degree of relaxation is increased. Thus, the congeniality estimation unit 20 may estimate that the congeniality Co between the first target person 110 and the second target person 120 is in the grade 1 (in the present example, the congeniality Co is good). If the second target person 120 is in the third state S2-3, it may be inferred that the second target person 120 is in the state where the irritation, nervousness, or stress is increased. Therefore, the congeniality estimation unit 20 may estimate that the congeniality Co between the first target person 110 and the second target person 120 is in the grade 2 (in the present example, the congeniality Co is poor). If the second target person 120 is in the fourth state S2-4, it may be inferred that the second target person 120 is in the state where the degree of preoccupation is increased. Thus, the congeniality estimation unit 20 may estimate that the congeniality Co between the first target person 110 and the second target person 120 is in the grade 1 (in the present example, the congeniality Co is good).

The situation where the first target person 110 is interacting with the second target person 120 is referred to as a situation Si. The situation Si refers to the interacting situation between the first target person 110 and the second target person 120 that may affect the state S1 of the first target person 110 or the state S2 of the second target person 120. The situation Si is, for example, the content of work, the content of conversation, or the like of the first target person 110 or the second target person 120.

The congeniality estimation unit 20 may estimate the congeniality Co based on the situation Si and the state S1 of the first target person 110. For example, if the congeniality estimation unit 20 estimates that the state S1 of the first target person 110 is the first state S1-1 or the third state S1-3 described above even in the situation Si that is unlikely to increase the stress state of the first target person 110, the probability of the congeniality Co being in the grade 2 (in the present example, the poor congeniality Co) is high. The situation Si that is unlikely to increase the stress state of the first target person 110 refers to the situation Si that is unlikely to affect the potential state S1 of the first target person 110. The situation Si refers to, for example, the situation where the first target person 110 simply communicates information to the second target person 120, or the like.

For example, if the congeniality estimation unit 20 estimates that the state S1 of the first target person 110 is the second state S1-2 or the fourth state S1-4 described above even in the situation Si that is likely to increase the stress state of the first target person 110, the probability of the congeniality Co being in the grade 1 (in the present example, the good congeniality Co) is high. The situation Si that is likely to increase the stress state of the first target person 110 refers to the situation Si that is likely to affect the potential state S1 of the first target person 110. The situation Si refers to, for example, the situation where the first target person 110 offers specific care, specific advice, or the like for the second target person 120. Similarly, the congeniality estimation unit 20 may estimate the congeniality Co based on the situation Si and the state S2 of the second target person 120.

In the case where at least one of the first target person 110 or the second target person 120 takes an action, at least one of each action of the first target person 110 or each action of the second target person 120 may be associated with at least one of each first electroencephalogram information Ib1 of the first target person 110 or each second electroencephalogram information Ib2 of the second target person 120. The action in the case where the first target person 110 takes an action refers to an action that may lead to the change in the potential state S1 of the first target person 110. The case where the first target person 110 takes an action refers to the case where, for example, when the first target person 110 is a caregiver and the second target person 120 is a care recipient, one first target person 110 goes to the location of each second target person 120 and interacts with each second target person 120. For example, if the first target person 110 performs clerical work alone, there is a high probability that the clerical work does not lead to the change in the potential state S1 of the first target person 110. Therefore, the case where such clerical work is performed or the like may not be the case where the first target person 110 takes an action.

The action in the case where the second target person 120 takes an action refers to an action that may lead to the change in the potential state S2 of the second target person 120. The case where the second target person 120 takes an action refers to the case where, for example, when one first target person 110 is a teacher of one subject, another first target person 110 is a teacher of another subject, and the second target person 120 is a student, the second target person 120 goes to the location of each first target person 110 and interacts with each first target person 110.

At least one of an action of each first target person 110 or an action of each second target person 120 and at least one of the first electroencephalogram information Ib1 of each first target person 110 or the second electroencephalogram information Ib2 of each second target person 120, which are associated with each other, may be recorded in the storage unit 50 as the time record (see FIG. 7). The time record is referred to as a time record Rt. Based on the time record Rt, the congeniality estimation unit 20 may determine the second target person 120 with which one first target person 110 was interacting in the case of one first electroencephalogram information Ib1.

In the case where one first target person 110 interacts with a plurality of second target persons 120, for example, if the proportion of the amplitude of the theta wave to the total amplitude As in the first electroencephalogram information Ib1 in the case where the one first target person 110 was interacting with one second target person 120 is greater than the proportion of the amplitude of the theta wave to the total amplitude As in the first electroencephalogram information Ib1 in the case where the one first target person 110 was interacting with another second target person 120, the congeniality estimation unit 20 can determine that the congeniality Co between the one first target person 110 and the one second target person 120 is less preferable than the congeniality Co between the one first target person 110 and the another second target person 120. Therefore, the congeniality estimation unit 20 can determine the second target person 120 who has less preferable congeniality Co with the one first target person 110.

The presentation unit 40 (see FIG. 7) may present a combination of the first target person 110 and the second target person 120 based on the determination result of the second target person 120 determined by the congeniality estimation unit 20. For example, if it is determined that the congeniality Co between one first target person 110 and one second target person 120 is less preferable than the congeniality Co between the one first target person 110 and another second target person 120, the presentation unit 40 may present the combination of the one first target person 110 and the another second target person 120 as the recommended combination.

Based on the time record Rt, the congeniality estimation unit 20 may determine the first target person 110 with which one second target person 120 was interacting in the case of one second electroencephalogram information Ib2. In the case where one second target person 120 interacts with a plurality of first target persons 110, for example, if the proportion of the amplitude of the theta wave to the total amplitude As in the second electroencephalogram information Ib2 in the case where the one second target person 120 was interacting with one first target person 110 is greater than the proportion of the amplitude of the theta wave to the total amplitude As in the second electroencephalogram information Ib2 in the case where the one second target person 120 was interacting with another first target person 110, the congeniality estimation unit 20 can determine that the congeniality Co between the one first target person 110 and the one second target person 120 is less preferable than the congeniality Co between the another first target person 110 and the one second target person 120. Therefore, the congeniality estimation unit 20 can determine the first target person 110 who has a less preferable congeniality Co with the one second target person 120.

The presentation unit 40 (see FIG. 7) may present the combination of the first target person 110 and the second target person 120 based on the determination result of the first target person 110 determined by the congeniality estimation unit 20. For example, if it is determined that the congeniality Co between one first target person 110 and one second target person 120 is less preferable than the congeniality Co between another first target person 110 and the one second target person 120, the presentation unit 40 may present the combination of the another first target person 110 and the one second target person 120 as the recommended combination.

The information acquisition unit 10 may further acquire the duration of the state S2 of the second target person 120. The information acquisition unit 10 may further acquire one duration T of a plurality of states S2 of the second target person 120. For example, the information acquisition unit 10 acquires the duration T of the third state S2-3 (see FIG. 10).

The congeniality estimation unit 20 may determine at least one second target person 120 with the duration T exceeding a predetermined duration threshold. If the state S2 is the first state S2-1 or the third state S2-3, the duration threshold may be the duration T beyond which there would be a high probability that undesirable symptoms occur in the second target person 120. The undesirable symptoms refer to, for example, the symptom associated with mental or physical disorders such as a symptom of depression, a symptom of neurosis, or the like.

The presentation unit 40 may present, to the administrator 130 (see FIG. 1), at least one second target person 120 for which estimation is performed by the congeniality estimation unit 20. Thus, the administrator 130 easily performs treatment such as counseling for the at least one second target person 120.

The information acquisition unit 10 may acquire the time at which one state S2 changes to another state S2. The congeniality estimation unit 20 may estimate the congeniality Co based on the temporal change from one state S2 to another state S2. The congeniality estimation unit 20 estimates the congeniality Co based on the temporal change, for example, from a third state S2-3 to a fourth state S2-4. In the case where one first target person 110 is interacting with one second target person 120, the time at which the one second target person 120 changes from the third state S2-3 to the fourth state S2-4 is referred to as a time T1. In the case where another first target person 110 is interacting with one second target person 120, the time at which the one second target person 120 changes from the third state S2-3 to the fourth state S2-4 is referred to as a time T2.

If the time T1 is shorter than the time T2, the congeniality estimation unit 20 may estimate that the congeniality Co between one first target person 110 and one second target person 120 is better than the congeniality Co between another first target person 110 and the one second target person 120. If the first target person 110 is a caregiver and the second target person 120 is a care recipient, the second target person 120 sometimes feels stress toward any first target person 110 when the care starts. However, the stress of the first target person 110 is sometimes eliminated sooner when the second target person 120 with preferable congeniality Co provides the care than when the second target person 120 with non-preferable congeniality Co provides the care. In the present example, the congeniality estimation unit 20 estimates the congeniality Co based on the temporal change from the one state S2 to the other state S2. Therefore, the congeniality estimation unit 20 can estimate the second target person 120 with preferable congeniality Co.

If one second target person 120 is estimated to be in one state S2 in the situation Si where one first target person 110 is interacting with the one second target person 120 and another second target person 120 is estimated to be in the one state S2 in the situation Si where one first target person 110 in interacting with another second target person 120, the presentation unit 40 (see FIG. 7) may present the one first target person 110. If the one state S2 is, for example, the third state S2-3 (see FIG. 10), there is a high probability that the way in which the one first target person 110 interacts with the second target person 120 has a problem. The presentation unit 40 presents one first target person 110 for the administrator 130 (see FIG. 1) so that the administrator 130 easily performs the treatment such as counseling for the one first target person 110.

If one first target person 110 is estimated to be in one state S1 in the situation Si where one first target person 110 is interacting with one second target person 120 and other first target person 110 is estimated to be in one state S1 in the situation Si where another first target person 110 is interacting with one second target person 120, the presentation unit 40 (see FIG. 7) may present one second target person 120. If one state S1 is, for example, the third state S1-3 (see FIG. 9), there is a high probability that the way in which one second target person 120 interacts in response to the contact from the first target person 110 has a problem. The presentation unit 40 presents one second target person 120 to the administrator 130 (see FIG. 1) so that the administrator 130 easily performs the treatment such as counseling for the one second target person 120.

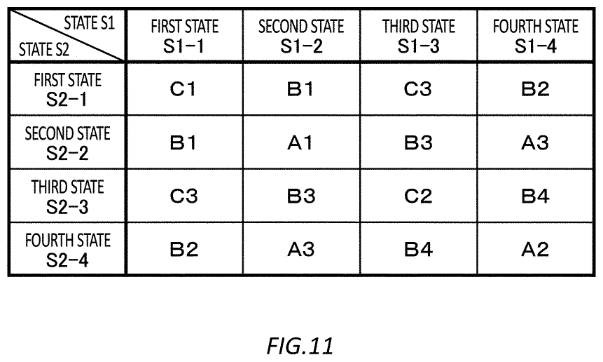

FIG. 11 is a diagram showing one example of the congeniality Co. The congeniality estimation unit 20 may estimate the congeniality Co between each first target person 110 and each second target person 120 based on the state S1 and the state S2. The congeniality estimation unit 20 may estimate the congeniality Co based on the combination of a plurality of states S1 and a plurality of states S2. In the present example, the congeniality Co is classified into A1-A3, B1-B4, and C1-C3. In the present example, the congeniality estimation unit 20 estimates the congeniality Co based on the first state S1-1 to the fourth state S1-4 of the first target person 110 and the first state S2-1 to the fourth state S2-4 of the second target person 120.

A1 is the case where the first target person 110 is in the second state S1-2 and the second target person 120 is in the second state S2-2. A2 is the case where the first target person 110 is in the fourth state S1-4 and the second target person 120 is in the fourth state S2-4. A1 is the case where the first target person 110 and the second target person are both in the state of a high degree of reassurance. A2 is the case where the first target person 110 and the second target person are both in the state of a high degree of interest. Therefore, in the case of A1 and A2, there is a high probability that the congeniality Co is good. A3 is the case where the first target person 110 is in the second state S1-2 and the second target person 120 is in the fourth state S2-4 or the case where the first target person 110 is in the fourth state S1-4 and the second target person 120 is in the second state S2-2.

B1 is the case where the first target person 110 is in the second state S1-2 and the second target person 120 is in the first state S2-1 or the case where the first target person 110 is in the first state S1-1 and the second target person 120 is in the second state S2-2. There may be the case where one of the first target person 110 and the second target person 120 is in the state of a high degree of reassurance while the other of the first target person 110 and the second target person 120 is in the state of a low degree of interest. For example, in the case where the first target person 110 unilaterally likes the second target person 120, the first target person 110 may be in the second state S1-2 and the second target person 120 may be in the first state S2-1. B1 is the case where the first target person 110 and the second target person 120 have such congeniality Co.

B2 is the case where the first target person 110 is in the fourth state S1-4 and the second target person 120 is in the first state S2-1 or the case where the first target person 110 is in the first state S1-1 and the second target person 120 is in the fourth state S2-4. There may be the case where one of the first target person 110 and the second target person 120 is in the state of a high degree of interest while the other of the first target person 110 and the second target person 120 is in the state of a low degree of interest. For example, in the case where the first target person 110 unilaterally shows an interest in the second target person 120, the first target person 110 may be in the fourth state S1-4 and the second target person 120 may be the first state S2-1. B2 is the case where the first target person 110 and the second target person 120 have such congeniality Co.

B3 is the case where the first target person 110 is in the third state S1-3 and the second target person 120 is in the second state S2-2 or the case where the first target person 110 is in the second state S1-2 and the second target person 120 is in the third state S2-3. There may be the case where one of the first target person 110 and the second target person 120 is in the state of a high degree of alertness while the other of the first target person 110 and the second target person 120 is in the state of a high degree of reassurance. For example, in the case where the first target person 110 unilaterally likes the second target person 120, the first target person 110 may be in the second state S1-2 and the second target person 120 may be in the third state S2-3. B3 is the case where the first target person 110 and the second target person 120 have such congeniality Co.

B4 is the case where the first target person 110 is in the fourth state S1-4 and the second target person 120 is in the third state S2-3 or the case where the first target person 110 is in the third state S1-3 and the second target person 120 is in the fourth state S2-4. There may be the case where one of the first target person 110 and the second target person 120 is in the state of a high degree of alertness while the other of the first target person 110 and the second target person 120 is in the state of a high degree of interest. For example, in the case where the first target person 110 unilaterally shows an interest in the second target person 120, the first target person 110 may be in the fourth state S1-4 and the second target person 120 may be in the third state S2-3. B4 is the case where the first target person 110 and the second target person 120 have such congeniality Co.

C1 is the case where the first target person 110 is in the first state S1-1 and the second target person 120 is in the first state S2-1. C1 is the state where the first target person 110 and the second target person are both in the state of a low degree of interest. C2 is the case where the first target person 110 is in the third state S1-3 and the second target person 120 is in the third state S2-3. C2 is the state where the first target person 110 and the second target person are both in the state of a high degree of alertness. Therefore, in the case of C1 and C2, there may be a high probability that the congeniality Co is poor. C3 is the case where the first target person 110 is in the first state S1-1 and the second target person 120 is in the third state S2-3 or the case where the first target person 110 is in the third state S1-3 and the second target person 120 is in the first state S2-1.

The congeniality estimation unit 20 may estimate which of A1-A3, B1-B4, and C1-C3 the congeniality Co is. It is noted that A1-A3, B1-B4, and C1-C3 are merely classifications of the congeniality Co and do not mean the level of the congeniality Co between the first target person 110 and the second target person 120.

The presentation unit 40 may present, to the administrator 130 (see FIG. 1), one grade estimated by the congeniality estimation unit 20. Thus, the administrator 130 easily performs treatment such as counseling for at least one of the first target person 110 or the second target person 120 according to each grade of the congeniality Co.

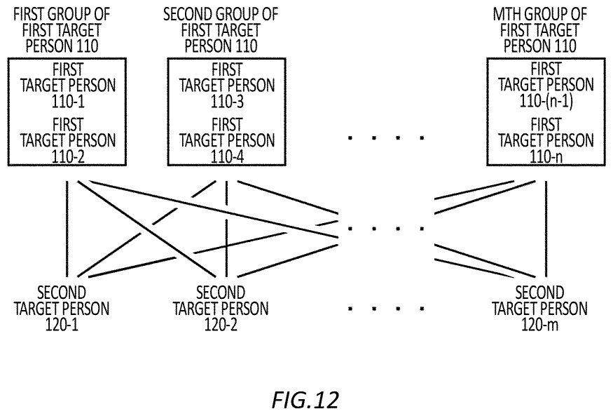

FIG. 12 is a diagram showing one example of a combination of the plurality of groups including two or more first target persons 110 and the plurality of second target persons 120. In the present example, one group of the first target persons 110 includes two first target persons 110. In the present example, there are m groups of first target persons 110 and there are m individuals of the second target person 120 (the second target person 120-1 to the second target person 120-m). Each of the first group to the mth group of the first target person 110 interacts with each of the second target person 120-1 to the second target person 120-m. In FIG. 12, the combinations of each group of the plurality of first target persons 110 and each of the plurality of second target persons 120 are indicated by solid lines.

The information acquisition unit 10 may acquire the second electroencephalogram information Ib2 in the situation Si where each group of the plurality of first target persons 110 is interacting with each of the second target persons 120. The congeniality estimation unit 20 may estimate the congeniality Co between each of the plurality of groups of the first target persons 110 and each of the second target persons 120 based on the second electroencephalogram information Ib2. For example, in the case where the first target person 110 is a caregiver and the second target person 120 is a care recipient, the second electroencephalogram information Ib2 in the case where each of one first target person 110 and another first target person 110 included in one group independently interacts with the second target person 120 may be different from the second electroencephalogram information Ib2 in the case where the one first target person 110 and the another first target person 110 simultaneously interact with the second target person 120 as one group. In the present example, the congeniality estimation unit 20 can estimate the congeniality Co between the group of the first target person 110 and the second target person 120.

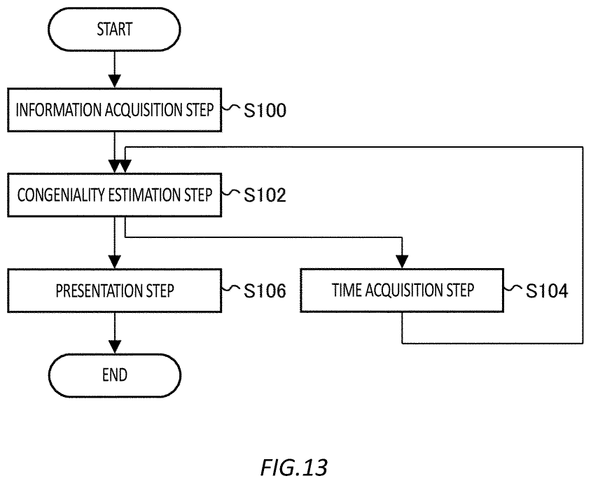

FIG. 13 is the flowchart showing one example of the congeniality estimation method according to one embodiment of the present invention. The congeniality estimation method includes the information acquisition step S100 and the congeniality estimation step S102. The congeniality estimation method may include the time acquisition step S104 and the presentation step S106. The congeniality estimation method according to one embodiment of the present invention is described using the congeniality estimation device 100 shown in FIG. 7 as an example.

The information acquisition step S100 is a step of acquiring, by the information acquisition unit 10, at least one of the first electroencephalogram information Ib1 of the plurality of first target persons 110 with the first attribute or the second electroencephalogram information Ib2 of the plurality of second target persons 120 with the second attribute in the situation Si where each of the first target persons 110 is interacting with each of the second target persons 120. The congeniality estimation step S102 is a step of estimating, by the congeniality estimation unit 20, the congeniality Co between each first target person 110 and each second target person 120 based on at least one of the first electroencephalogram information Ib1 or the second electroencephalogram information Ib2.

The information acquisition step S100 may be a step of further acquiring, by the information acquisition unit 10, at least one of the first biological information Ig1 of the first target person 110 or the second biological information Ig2 of the second target person 120 in the situation Si where each first target person 110 is interacting with each second target person 120. The congeniality estimation step S102 may be a step of estimating, by the congeniality estimation unit 20, the congeniality Co based on at least one of the first electroencephalogram information Ib1 or the second electroencephalogram information Ib2 and at least one of the first biological information Ig1 or the second biological information Ig2.

The information acquisition step S100 may be a step of further acquiring, by the information acquisition unit 10, the third electroencephalogram information Ib3 of the first target person 110 before each first target person 110 interacts with each second target person 120. The congeniality estimation step S102 may be a step of estimating, by the congeniality estimation unit 20, the congeniality Co based on the change from the third electroencephalogram information Ib3 to the first electroencephalogram information Ib1 and the first biological information Ig1.

The congeniality estimation step S102 may be a step of estimating, by the congeniality estimation unit 20, the congeniality Co based on the change (the change C1 described above) from the proportion of the amplitude Af1 to the total amplitude As to the proportion of the amplitude Af2 to the total amplitude As and the ratio of LF1 to HF1 (LF1/HF1). The congeniality estimation step S102 may be a step of estimating, by the congeniality estimation unit 20, the congeniality Co based on the magnitude relationship between the ratio Rag1 and the threshold Pth1, and the change C1.

The congeniality estimation step S102 may be a step where the congeniality estimation unit 20 estimates the state S1 of the first target person 110 based on the first electroencephalogram information Ib1 and the first biological information Ig1, and then estimates the congeniality Co based on the situation Si where each first target person 110 is interacting with each second target person 120 and the state S1.

The congeniality estimation step S102 may be a step where the congeniality estimation unit 20 estimates the state S1 of the first target person 110 based on the first electroencephalogram information Ib1 and the first biological information Ig1, estimates the state S2 of the second target person 120 based on the second electroencephalogram information Ib2 and the second biological information Ig2, and then estimates the congeniality Co based on the state S1 of the first target person 110 and the state S2 of the second target person 120.

The congeniality estimation step S102 may be a step where the congeniality estimation unit 20 determines the second target person 120 with which one first target person 110 is interacting in the case of one first electroencephalogram information Ib1 or determines the first target person 110 with which one second target person 120 is interacting in the case of one second electroencephalogram information Ib2, based on at least one of the action of each first target person 110 or the action of each second target person 120 and the time record Rt in which at least one of the first electroencephalogram information Ib1 or the second electroencephalogram information Ib2 was recorded.

The presentation step S106 may be a step of presenting, by the presentation unit 40, the combination of the first target person 110 and the second target person 120 based on the determination result of the second target person 120 or the determination result of the first target person 110 determined in the congeniality estimation step S102.

In the case where each of a plurality of groups including two or more first target persons 110 interacts with each second target person 120, the information acquisition step S100 may be a step of acquiring, by the information acquisition unit 10, the second electroencephalogram information Ib2 in the situation Si where each of plurality of groups is interacting with each second target person 120. The congeniality estimation step S102 may be a step of estimating, by the congeniality estimation unit 20, the congeniality Co between each of the plurality of groups and each second target person 120 based on the second electroencephalogram information Ib2.

The congeniality estimation step S102 may be a step of estimating, by the congeniality estimation unit 20, the state S2 of the second target person 120 based on the second electroencephalogram information Ib2 and the second biological information Ig2. The time acquisition step S104 may be a step of acquiring, by the information acquisition unit 10, the duration T of the state S2 of the second target person 120. The congeniality estimation step S102 may be a step of determining, by the congeniality estimation unit 20, at least one second target person 120 with the duration T exceeding a predetermined duration threshold.

The congeniality estimation step S102 may be a step of estimating, by the congeniality estimation unit 20, a plurality of states S2 of the second target person 120 based on the second electroencephalogram information Ib2 and the second biological information Ig2. The time acquisition step S104 may be a step of acquiring, by the information acquisition unit 10, the time at which one state S2 changes to another state S2. The congeniality estimation step S102 may be a step of estimating, by the congeniality estimation unit 20, the congeniality Co based on the temporal change from one state S2 to another state S2.

The congeniality estimation step S102 may be a step of estimating, by the congeniality estimation unit 20, one of a plurality of states S2 of the second target person 120 based on the second electroencephalogram information Ib2 and the second biological information Ig2. The presentation step S106 may be a step of presenting, by the presentation unit 40, one first target person 110 if, in the situation Si where one first target person 110 is interacting with one second target person 120, it is estimated that one second target person 120 is in one state S2 and, in the situation Si where one first target person 110 is interacting with another second target person 120, it is estimated that another second target person 120 is in the one state S2.

The congeniality estimation step S102 may be a step of estimating, by the congeniality estimation unit 20, one of the plurality of states S1 of the first target person 110 based on the first electroencephalogram information Ib1 and the first biological information Ig1. The presentation step S106 may be a step of presenting, by the presentation unit 40, one second target person 120 if, in the situation Si where one first target person 110 is interacting with one second target person 120, it is estimated that one first target person 110 is in one state S1 and, in the situation Si where another first target person 110 is interacting with one second target person 120, it is estimated that another first target person 110 is in the one state S1.

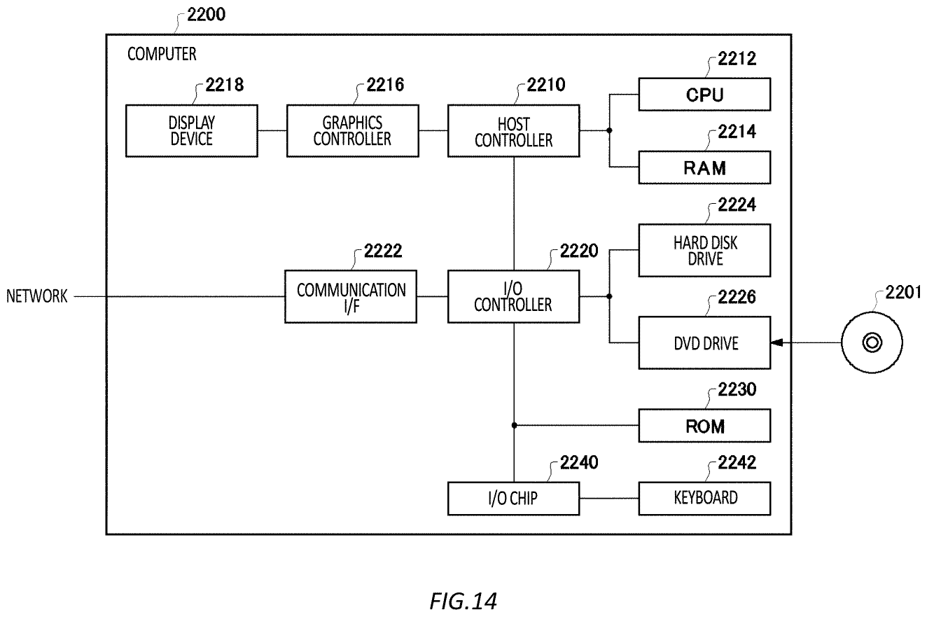

FIG. 14 is a diagram showing one example of a computer 2200 in which the congeniality estimation device 100 according to one embodiment of the present invention may be wholly or partially embodied. The program installed on the computer 2200 can cause the computer 2200 to function as an operation associated with the congeniality estimation device 100 according to the embodiment of the present invention or one or more sections of the congeniality estimation device 100, or to perform the operation or the one or more sections, or can cause the computer 2200 to perform each step according to the congeniality estimation method of the present invention (see FIG. 10). Such a program may be executed by a CPU 2212 to cause a computer 2200 to execute certain operations associated with some or all of the blocks of flowcharts (refer to FIG. 13) and block diagrams (refer to FIG. 7) described herein.

The computer 2200 according to one embodiment of the present invention includes the CPU 2212, a RAM 2214, a graphics controller 2216, and a display device 2218. The CPU 2212, the RAM 2214, the graphics controller 2216, and the display device 2218 are mutually connected by a host controller 2210. The computer 2200 further includes input/output unit such as a communication interface 2222, a hard disk drive 2224, a DVD-ROM drive 2226, and an IC card drive. The communication interface 2222, the hard disk drive 2224, the DVD-ROM drive 2226, and the IC card drive, and the like are connected to the host controller 2210 via an input/output controller 2220. The computer further includes legacy input/output units such as a ROM 2230 and a keyboard 2242. The ROM 2230, the keyboard 2242, and the like are connected to the input/output controller 2220 via an input/output chip 2240.

The CPU 2212 operates according to programs stored in the ROM 2230 and the RAM 2214, thereby controlling each unit. The graphics controller 2216 acquires image data generated by the CPU 2212 on a frame buffer or the like provided in the RAM 2214 or in the RAM 2214 itself to cause the image data to be displayed on the display device 2218.

The communication interface 2222 communicates with other electronic devices via a network. The hard disk drive 2224 stores programs and data used by the CPU 2212 in the computer 2200. The DVD-ROM drive 2226 reads the programs or the data from the DVD-ROM 2201, and provides the read programs or data to the hard disk drive 2224 via the RAM 2214. The IC card drive reads programs and data from an IC card, or writes programs and data to the IC card.

The ROM 2230 stores a boot program or the like executed by the computer 2200 at the time of activation, or a program depending on the hardware of the computer 2200. The input/output chip 2240 may connect various input/output unit via a parallel port, a serial port, a keyboard port, a mouse port, or the like to the input/output controller 2220.

Programs are provided by a computer readable medium such as the DVD-ROM 2201 or the IC card. The programs are read from the computer readable medium, are installed in the hard disk drive 2224, the RAM 2214, or the ROM 2230 which is also an example of the computer readable medium, and are executed by the CPU 2212. The information processing described in these programs is read by the computer 2200, and provides cooperation between the programs and the various types of hardware resources. An apparatus or method may be constituted by realizing the operation or processing of information in accordance with the usage of the computer 2200.

For example, when a communication is executed between the computer 2200 and an external device, the CPU 2212 may execute a communication program loaded onto the RAM 2214 to instruct communication processing to the communication interface 2222, based on the processing described in the communication program. The communication interface 2222, under control of the CPU 2212, reads transmission data stored on a transmission buffering region provided in a recording medium such as the RAM 2214, the hard disk drive 2224, the DVD-ROM 2201, or the IC card, and transmits the read transmission data to a network or writes reception data received from a network to a reception buffering region or the like provided on the recording medium.

The CPU 2212 may cause all or a necessary portion of a file or a database to be read into the RAM 2214, the file or the database having been stored in an external recording medium such as the hard disk drive 2224, the DVD-ROM drive 2226 (DVD-ROM 2201), the IC card, or the like. The CPU 2212 may execute various types of processing on the data on the RAM 2214. The CPU 2212 may then write back the processed data to the external recording medium.

Various types of information, such as various types of programs, data, tables, and databases, may be stored in the recording medium to undergo information processing. The CPU 2212 may execute various types of processing on the data read from the RAM 2214, which includes various types of operations, information processing, condition judging, conditional branch, unconditional branch, search or replace of information, or the like, as described throughout the present disclosure and designated by an instruction sequence of programs. The CPU 2212 may write the result back to the RAM 2214.

The CPU 2212 may search for information in a file, a database, or the like in the recording medium. For example, when a plurality of entries, each having an attribute value of a first attribute associated with an attribute value of a second attribute, are stored in the recording medium, the CPU 2212 may search for an entry matching the condition whose attribute value of the first attribute is designated, from among the plurality of entries, read the attribute value of the second attribute stored in the entry, and read a second attribute value to acquire the attribute value of the second attribute associated with the first attribute satisfying the predetermined condition.

The program or software modules described above may be stored in the computer readable media on the computer 2200 or of the computer 2200. A recording medium such as a hard disk or a RAM provided in a server system connected to a dedicated communication network or the Internet can be used as the computer readable media. The program may be provided to the computer 2200 by the recording medium.

While the present invention has been described by way of the embodiments, the technical scope of the present invention is not limited to the scope described in the above-described embodiments. It is apparent to persons skilled in the art that various alterations or improvements can be made to the above-described embodiments. It is also apparent from the described scope of the claims that the embodiments added with such alterations or improvements can be included the technical scope of the present invention.

The operations, procedures, steps, stages, or the like of each process performed by a device, system, program, and method shown in the claims, a specification, or drawings can be performed in any order as long as the order is not indicated by “prior to,” “before,” or the like and as long as the output from a previous process is not used in a later process. Even if the process flow is described using phrases such as “first” or “next” for convenience in the claims, a specification, or drawings, it does not necessarily mean that the process must be performed in this order.

### EXPLANATION OF REFERENCES

10 information acquisition unit,
20 congeniality estimation unit,
40 presentation unit,
50 storage unit,
100 congeniality estimation device,
110 first target person,
120 second target person,
130 administrator,
2200 computer,
2201 DVD-ROM,
2210 host controller,
2212 CPU,
2214 RAM,
2216 graphics controller,
2218 display device,
2220 input/output controller,
2222 communication interface,
2224 hard disk drive,
2226 DVD-ROM drive,
2230 ROM,
2240 input/output chip,
2242 keyboard.

What is claimed is:

1. A congeniality estimation device comprising:
an information acquisition unit that acquires at least one of first electroencephalogram information of a plurality of first target persons with a first attribute or second electroencephalogram information of a plurality of second target persons with a second attribute in a situation where each of the first target persons is interacting with each of the second target persons; and
a congeniality estimation unit that estimates congeniality between each of the first target persons and each of the second target persons based on at least one of the first electroencephalogram information or the second electroencephalogram information.

2. The congeniality estimation device according to claim 1, wherein the information acquisition unit further acquires at least one of first biological information of the first target persons or second biological information of the second target persons in a situation where each of the first target persons is interacting with each of the second target persons, and
the congeniality estimation unit estimates the congeniality based on at least one of the first electroencephalogram information or the second electroencephalogram information and at least one of the first biological information or the second biological information.

3. The congeniality estimation device according to claim 2, wherein the information acquisition unit further acquires third electroencephalogram information of the first target persons before each of the first target persons interacts with each of the second target persons, and
the congeniality estimation unit estimates the congeniality based on a change from the third electroencephalogram information to the first electroencephalogram information and the first biological information.

4. The congeniality estimation device according to claim 3, wherein the congeniality estimation unit estimates the congeniality based on a change from a proportion of an amplitude of an electroencephalogram in a predetermined frequency band to a total amplitude in the third electroencephalogram information to a proportion of an amplitude of an electroencephalogram in the frequency band to the total amplitude in the first electroencephalogram information and a ratio of a magnitude of a first power spectrum to a magnitude of a second power spectrum in a heartbeat of the first target persons,
the total amplitude is a sum of amplitudes of an alpha wave, a beta wave, a theta wave, a gamma wave, and a delta wave, and
a frequency band of the second power spectrum is a higher frequency band than a frequency band of the first power spectrum.

5. The congeniality estimation device according to claim 4, wherein the congeniality estimation unit estimates the congeniality based on a ratio of a magnitude of the first power spectrum to a magnitude of the second power spectrum, a magnitude relationship between the ratio of the magnitude of the first power spectrum to the magnitude of the second power spectrum and a predetermined threshold, and the change from the ratio in the third electroencephalogram information to the ratio in the first electroencephalogram information.

6. The congeniality estimation device according to claim 2, wherein the congeniality estimation unit estimates a state of the first target persons based on the first electroencephalogram information and the first biological information and estimates the congeniality based on the situation where each of the first target persons is interacting with each of the second target persons and the state.

7. The congeniality estimation device according to claim 3, wherein the congeniality estimation unit estimates a state of the first target persons based on the first electroencephalogram information and the first biological information and estimates the congeniality based on the situation where each of the first target persons is interacting with each of the second target persons and the state.

8. The congeniality estimation device according to claim 2, wherein the congeniality estimation unit estimates a state of the first target persons based on the first electroencephalogram information and the first biological information, estimates a state of the second target persons based on the second electroencephalogram information and the second biological information, and estimates the congeniality based on the state of the first target persons and the state of the second target persons.

9. The congeniality estimation device according to claim 3, wherein the congeniality estimation unit estimates a state of the first target persons based on the first electroencephalogram information and the first biological information, estimates a state of the second target persons based on the second electroencephalogram information and the second biological information, and estimates the congeniality based on the state of the first target persons and the state of the second target persons.

10. The congeniality estimation device according to claim 1, wherein the congeniality estimation unit determines, based on a time record in which at least one of an action of each of the first target persons or an action of each of the second target persons and at least one of the first electroencephalogram information or the second electroencephalogram information are recorded, the second target persons with which one of the first target persons is interacting in a case of one piece of the first electroencephalogram information or determines the first target persons with which one of the second target persons is interacting in a case of one piece of the second electroencephalogram information.

11. The congeniality estimation device according to claim 2, wherein the congeniality estimation unit determines, based on a time record in which at least one of an action of each of the first target persons or an action of each of the second target persons and at least one of the first electroencephalogram information or the second electroencephalogram information are recorded, the second target persons with which one of the first target persons is interacting in a case of one piece of the first electroencephalogram information or determines the first target persons with which one of the second target persons is interacting in a case of one piece of the second electroencephalogram information.

12. The congeniality estimation device according to claim 10, further comprising a presentation unit that presents a combination of the first target persons and the second target persons based on a determination result of the second target persons or a determination result of the first target persons that is determined by the congeniality estimation unit.

13. The congeniality estimation device according to claim 1, wherein, when each of a plurality of groups including two or more of the first target persons is interacting with each of the second target persons, the information acquisition unit acquires the second electroencephalogram information in the situation where each of the plurality of groups is interacting with each of the second target persons, and
the congeniality estimation unit estimates the congeniality between each of the plurality of groups and each of the second target persons based on the second electroencephalogram information.

14. The congeniality estimation device according to claim 2, wherein, when each of a plurality of groups including two or more of the first target persons is interacting with each of the second target persons, the information acquisition unit acquires the second electroencephalogram information in the situation where each of the plurality of groups is interacting with each of the second target persons, and
the congeniality estimation unit estimates the congeniality between each of the plurality of groups and each of the second target persons based on the second electroencephalogram information.

15. The congeniality estimation device according to claim 2, wherein the congeniality estimation unit estimates a state of the second target persons based on the second electroencephalogram information and the second biological information,
the information acquisition unit further acquires a duration of the state of the second target persons, and
the congeniality estimation unit determines at least one of the second target persons with the duration exceeding a predetermined duration threshold.

16. The congeniality estimation device according to claim 2, wherein the congeniality estimation unit estimates a plurality of states of the second target persons based on the second electroencephalogram information and the second biological information and estimates the congeniality based on a temporal change from one of the plurality of states to another of the plurality of states.

17. A congeniality estimation device according to claim 2, further comprising a presentation unit that presents the first target persons, wherein
the congeniality estimation unit estimates one of a plurality of states of the second target persons based on the second electroencephalogram information and the second biological information, and
if one of the second target persons is estimated to be in one of the plurality of states in the situation where one of the first target persons is interacting with the one of the second target persons, and another of the second target persons is estimated to be in the one of the plurality of states in the situation where the one of the first target persons is interacting with the another of the second target persons, the presentation unit presents the one of the first target persons.

18. The congeniality estimation device according to claim 2, further comprising a presentation unit that presents the second target persons, wherein
the congeniality estimation unit estimates one of a plurality of states of the first target persons based on the first electroencephalogram information and the first biological information, and
if one of the first target persons is estimated to be in one of the plurality of states in the situation where the one of the first target persons is interacting with one of the second target persons and another of the first target persons is estimated to be in the one of the plurality of states in the situation where the another of the first target persons is interacting with the one of the second target person, the presentation unit presents the one of the second target persons.

19. A congeniality estimation method comprising:
acquiring, by an information acquisition unit, at least one of first electroencephalogram information of a plurality of first target persons with a first attribute or second electroencephalogram information of a plurality of second target persons with a second attribute in a situation where each of the first target persons is interacting with each of the second target persons; and
estimating, by a congeniality acquisition unit, a congeniality between each of the first target persons and each of the second target persons based on at least one of the first electroencephalogram information or the second electroencephalogram information.

20. A non-transitory computer-readable medium that has a congeniality estimation program recorded thereon that, when executed by a computer, causes the computer to perform procedures of:
acquiring at least one of first electroencephalogram information of a plurality of first target persons with a first attribute or second electroencephalogram information of a plurality of second target persons with a second attribute in a situation where each of the first target persons is interacting with each of the second target persons; and
estimating a congeniality between each of the first target persons and each of the second target persons based on at least one of the first electroencephalogram information or the second electroencephalogram information.
